SEQUENCE LISTING (1 sequence)

SEQ ID NO: 1 - protein, 276 aa, Artificial Sequence
  Synthetic Peptide
MSRLDKSKVINSALELLNEVGIEGLTTRKLARKLGVEQPTLYWHVKNKRALLDALAIEMLDRHHTHFCPLEGESWQDFLRNNAKSFRRALLSHRDGAKVHLGTRPTEKQYETLENQLRFLCQQGFSLRNALYALSAVGHFTLGCVLEDQEHQVAKEERETPTTDSMPPLLRQAIELFDHQGAEPAFLFGLELIIRGLEKQLKCESGGPKLHNHNHNHNHNHNEFAAAAAAAAAGTPADALDDFDLDMLPADALDDFDLDMLPADALDDFDLDMLPG

(19) United States
(12) Patent Application Publication
(10) Pub. No.: US 2012/0190107 A1
(43) Pub. Date: Jul. 26, 2012

(54) ENHANCED PROTEIN TRANSDUCTION

(76) Inventors: Dwayne Bisgrove, Mountain View, CA (US); Hiroaki Sagawa, Mountain View, CA (US)

(21) Appl. No.: 13/358,450

(22) Filed: Jan. 25, 2012

Related U.S. Application Data
(60) Provisional application No. 61/436,495, filed on Jan. 26, 2011.

(51) Int. Cl. C12N 5/071; C12N 5/07; C12N 1/21; C12N 1/14; C12N 1/16; C12N 15/63; C12N 5/04

(52) U.S. Cl. 435/348; 435/320.1; 435/375; 435/350; 435/351; 435/354; 435/363; 435/366; 435/410; 435/254.1; 435/255.1; 435/255.2; 435/255.5; 435/252.1; 435/255.6

(57) ABSTRACT

Methods of enhanced protein transduction are provided. Aspects of the methods include contacting a cell with a transduction protein, where the transduction protein includes both a protein-of-interest domain and a protein transduction domain, and a nucleic acid transfection agent. Also provided are systems and kits that find use in practicing methods according to embodiments of the invention. The methods, systems and kits find use in a variety of different applications.

### CROSS REFERENCE To RELATED APPLICATIONS

Pursuant to 35 U.S.C. §119 (e), this application claims priority to the filing date of the U.S. Provisional Patent Application Ser. No. 61/436,495, filed Jan. 26, 2011; the disclosure of which is herein incorporated by reference.

### INTRODUCTION

Protein transduction domains (PTDs), also known as cell penetrating peptides, are a class of small peptides capable of penetrating the plasma membrane of mammalian cells (B. Godin, E. Touitou. “Mechanism of bacitracin permeation enhancement through the skin and cellular membranes from an ethosomal carrier,” J. Control. Rel. 94:365-379 (2004)). There are several well-known PTDs: the HIV transcription factor TAT, the Antp peptide derived from the Drosophila melanogaster homeodomain protein, the herpes simplex virus protein VP22, and arginine oligomers (Pillai & Panchagnula, Transdermal iontophoresis of insulin: IV. Influence of chemical enhancers, Int. J. Pharm. 269:109-120 (2004); Kalia et al., Iontophoretic drug delivery, Adv. Drug Deliv. Rev. 56:619-658 (2004); and S. Mitragotri, “Synergistic effect of enhancers for transdermal drug delivery,” Pharm. Res. 17:1354-1359 (2000)). These peptides have been reported to transport compounds of many types and molecular weights, such as conjugated peptides, oligonucleotides, and small particles such as liposomes across mammalian cells (Mitragotri, supra; Boinpally et al., Iontophoresis of lecithin vesicles of clyclosporin A, Int. J. Pharm. 274:185-190 (2004); Lindgren et al., Cell-penetrating peptides, Trends Pharmacol. Sci. 21:99-103 (2000); and Schwarze & Dowdy, In vivo protein transduction: intracellular delivery of biologically active proteins, compounds and DNA, Trends Pharmacol. Sci. 21:45-48 (2000). Thus, PTDs represent an important class of drug delivery approaches.

### SUMMARY

Methods of enhanced protein transduction are provided. Aspects of the methods include contacting a cell with a transduction protein, where the transduction protein includes both a protein-of-interest domain and a protein transduction domain, and a nucleic acid transfection agent. Also provided are systems and kits that find use in practicing methods according to embodiments of the invention. The methods, systems and kits find use in a variety of different applications.

### BRIEF DESCRIPTION OF THE FIGURES

FIG. 1 provides the results of a luciferase assay showing enhancement of Tet Express activity. HeLa reporter cells (Clone 19) with a stable bidirectional TREtight ZsGreen/FLuc integration cassette were plated in 96 well format (15,000 cells per well). After cell attachment, 0, 5, 10 or 20 μL of transduction mix were added per well. Transduction mixes consisted of Tet Express protein in Optimem media with one of the following additions: 2% LTX, 0.6% Xfect, 2% Lipofectamine 2000 (Lipo2000), 0.2 mM chloroquine or 6 μg/mL polybrene. Luciferase assays were performed the following day.

FIG. 2 provides the results of a luciferase assay showing enhancement of Tet Express activity. Clone 19 HeLa reporter cells were plated in 96 well format (15,000 cells per well). Tet Express protein (in Optimem media) was mixed with indicated amount of reagent and added to the cells. Reagents included Mirus' Transit-siQuest, Transit-TKO, Transit-LTI, Transit-Jurkat, Transit-2020 or Clontech's Xfect. Luciferase assays were performed the following day.

FIGS. 3A-3C. provide the results of a luciferase assay using heat inactivated Tet Express. Clone 19 HeLa reporter cells were plated in 96 well format (15,000 cells per well). Ten micrograms of Tet Express or heat inactivated Tet Express (75° C. for 5 minutes) was mixed with either: 0, 1, 2 or 4 μL of Xfect and added to the cells. Luciferase assays were performed the following day. FIG. 3B illustrates that DNase treatment does not alter Tet Express activity. Clone 19 HeLa reporter cells were plated in 96 well format (15,000 cells per well). Tet Express without or with DNase I pretreatment (10 units per 150 μL Tet Express, 37° C. for 30 minutes) was mixed with Xfect and added to cells. Luciferase assay was performed the following day. In FIG. 7C., Clone 19 HeLa reporter cells were plated in 12 well format (50,000 cells per well). Cells were transfected with 0.5 μg of pUC19 (control), prOF7, pTet On Advanced or pTet Off Advanced plasmids with 2 μL of Lipofectamine LTX. The following day doxycyline was added to 1000 ng/mL. Luciferase assays were performed the following day.

FIG. 4 illustrates the effect of Xfect on Transactivation. Clone 19 HeLa reporter cells were plated at 50,000 cells per well in a 12 well format. Cells were treated with 0, 1, 10 or 100 μg of Tet Express (or derivative) in the presence or absence of Xfect reagent. Luciferase assays were performed the following day.

### DETAILED DESCRIPTION

Methods of enhanced protein transduction are provided. Aspects of the methods include contacting a cell with a transduction protein, where the transduction protein includes both a protein-of-interest domain and a protein transduction domain, and a nucleic acid transfection agent. Also provided are systems and kits that find use in practicing methods according to embodiments of the invention. The methods, systems and kits find use in a variety of different applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods of Enhanced Protein Transduction

As summarized above, aspects of the invention include methods of protein transduction. “Protein transduction” as used here refers to the internalization from the external environment of proteins into a cell. As such, when a cell is transduced with a protein, the protein transits the cell membrane so as to pass from the external environment of the cell into the cell, e.g., into the cytoplasm of the cell. Protein transduction methods according to certain embodiments of the invention are enhanced. By enhanced is meant that protein transduction according to embodiments of the invention is 2-fold or greater more efficient, such as 5-fold or greater more efficient including 10-fold or greater more efficient as compared to protein transduction methods that do not include use of a nucleic acid transfection agent, e.g., as described in greater detail below.

The protein that is employed in protein transduction methods of the invention is referred to herein as the “transduction protein.” The transduction protein is the protein that crosses the cell membrane from the external environment of the cell into the cell during the protein transduction methods described herein. Transduction proteins include at least two components, which are a protein-of-interest component or domain (i.e., a POI domain) and a protein transduction domain. These two components or domains may be distinct or non-distinct, as described in greater detail below.

### Protein-of-Interest (POI) Domain

The POI domain of the transduction protein may be any peptide, polypeptide or protein. POIs of interest include research POIs, diagnostic POIs and therapeutic POIs. Research POIs are protein domains whose activity finds use in a research protocol. As such, research POIs are protein domains that are employed in an experimental procedure. The research POI may be any POI that has such utility, where in some instances the research POI is a protein domain that is also provided in research protocols by expressing it in a cell from an encoding vector. Examples of specific types of research POIs include, but are not limited to: transcription modulators of inducible expression systems, members of signal production systems, e.g., enzymes and substrates thereof, hormones, prohormones, proteases, enzyme activity modulators, perturbimers and peptide aptamers, antibodies, modulators ofprotein-protein interactions and the like.

Diagnostic POIs are protein domains whose activity finds use in a diagnostic protocol. As such, diagnostic POIs are protein domains that are employed in a diagnostic procedure. The diagnostic POI may be any POI that has such utility. Examples of specific types of diagnostic POIs include, but are not limited to: members of signal production systems, e.g., enzymes and substrates thereof, labeled binding members, e.g., labeled antibodies and binding fragments thereof, peptide aptamers and the like.

POIs of interest further include therapeutic POIs. Therapeutic POIs of interest include without limitation, hormones and growth and differentiation factors including, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor .alpha. (TGFα), platelet-derived growth factor (PDGF), insulin growth factors I and II (IGF-I and IGF-II), any one of the transforming growth factor .beta. superfamily, including TGFβ, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

POIs of interest further include, but are not limited to: fibrinolytic proteins, including without limitation, urokinase-type plasminogen activator (u-PA), and tissue plasminogen activator (tpA); procoagulant proteins, such as Factor VIIa, Factor VIII, Factor IX and fibrinogen; plasminogen activator inhibitor-1 (PAI-1), von Willebrand factor, Factor V, ADAMTS-13 and plasminogen for use in altering the hemostatic balance at sites of thrombosis; etc.

Also of interest as POIs are transcription factors such as jun, fos, max, mad, serum response factor (SRF), AP-1, AP2, myb, MyoD, myogenin, ETS-box containing proteins, TFE3, E2F, ATF1, ATF2, ATF3, ATF4, ZF5, NFAT, CREB, HNF4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Also of interest as POIs are carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence.

In certain embodiments POI are to be understood to also include non-covalently bound complexes. These can be protein-protein complexes as well as protein-mRNA, protein-non-coding RNA, protein-lipid and protein-small molecule complexes. Examples of such complexes are RISCs, spliceosomes, etc.

### Protein Transduction Domain (PTD)

In methods of the invention, the transduction proteins employed to transduce cells are ones that include both a POI domain and a protein transduction domain, as reviewed above. The PTD is a domain that confers onto the protein the ability to cross the cell membrane and therefore enable the protein to be internalized by the cell. The PTD may vary, where PTDs of interest include both distinct PTDs and distributed PTDs, e.g., as described in greater detail below.

### Distinct PTDs

As mentioned above, in some instances the PTD is a distinct PTD. Distinct PTDs are PTDs that make up a defined location or domain of the transduction protein, e.g., a terminal domain, such as an N- or C-terminal domain, or a central domain. Distinct PTDs are different from distributed PTDs (described in greater detail below) as they are not interspersed with other domains of the transduction protein, such as the POI portion. As such, transduction proteins that include distinct PTDs can be viewed as fusion proteins that include the POI as a first domain and the PTD as a second domain, where the two domains are encoded by separate regions of an encoding nucleic acid and arranged relative to each other in any order on a protein, such that the PTD domain may be N-terminal to the POI domain or vice versa.

Distinct PTDs may include short cationic peptides that can bind to the cell surface through electrostatic attachment to the cell membrane and can be uptaken by the cell by membrane translocation (Kabouridis (2003) TRENDS Biotech 21(11) 498- 503). PTDs of interest may interact with a target cell via binding to glycosaminoglycans (GAGs), such as for example, hyaluronic acid, heparin, heparan sulfate, dermatan sulfate, keratin sulfate or chondroitin sulfate and their derivatives. Distinct PTDs can be of any length. In some instances, the length of the PTD ranges from 5 to 100 amino acids, such as from 5 to 25 amino acids, where in some instances, the PTD is 5, 6, 7, 8, 9, 10, 11, 12, 13, 14, 15, 16, 17, 18, 19, 20, 21, 22, 23, 24 or 25 amino acids in length. A given transduction protein molecule of the invention may include a single PTD or multiple PTDs. For example, multiple copies of the same PTD (e.g., dimer, trimer, tetramer, pentamer, hexamer, heptamer, octamer, nonamer, decamer or larger multimer) or different PTDs can be conjugated to the POI domain of the transduction protein to make the transduction protein molecule.

Specific PTDs of interest include, but are not limited to, PTDs derived from human immunodeficiency virus 1 (HIV-I) TAT (Ruben et al. (1989) J. Virol. 63:1-8); the herpes virus tegument protein VP22 (Elliott and O'Hare (1997) Cell 88:223-233); the homeotic protein of Drosophila melanogaster Antennapedia (Antp) protein (Penetratin PTD, Derossi et al. (1996) J. Biol. Chem. 271 :18188-18193); the protegrin 1 (PG-I) antimicrobial peptide SynB (Kokryakov et al. (1993) FEBS Lett. 327:231-236); and the Kaposi fibroblast growth factor (Lin et al., (1995) J. Biol. Chem. 270-14255-14258). Also of interest as PTDs are synthetic PTDs. Synthetic PTDs of interest include, but are not limited to: transportan (Pooga et al. (1988) FASEB J. 12:67-77; Pooga et al. (.2001) FASEB J. 15:1451-1453), MAP (Oehlke et al. (1998) Biochim. Biophys. Acta. 1414: 127-139), KALA (Wyman et al. (1997) Biochemistry 36:3008-3017) and other cationic peptides, such as, for example, various β-cationic peptides (Akkarawongsa et al. (2008) Antimicrob. Agents and Chemother. 52(6):2120-2129). Additional PTD peptides and variant PTDs also are provided in, for example, U.S. Patent Publication Nos. US 2005/0260756, US 2006/0178297, US 2006/0100134, US 2006/0222657, US 2007/0161595, US 2007/0129305, European Patent Publication No. EP 1867661, PCT Publication Nos. WO 2000/062067, WO 2003/035892, WO 2007/097561 and WO 2007/053512, the disclosure of the PTDs in these references being incorporated herein by reference. Also of interest as PTDs are those that have TAT-like transduction domains, prion-like transduction domains (Wadia et al. (2008) PLoS ONE 3(10) e3314: 1-8), and transduction domains with basic charges clustered on one face of the peptide alpha-helix. For example, the PTDs of interest include, but not limited to, those disclosed in W02010/129033; the disclosure of which PTDs are herein incorporated by reference.

Transduction proteins that include a distinct PTD may be fabricated using any convenient protocol. The PTD can be conjugated to the POI using any convenient protocol, such as, for example, conjugation by recombinant means or by chemical coupling. The linkage of the components in the conjugate can be by any convenient method, so long as the attachment of the linker moiety to the POI does not substantially impede the desired activity of the POI.

Any convenient linker may be employed. Linkers and linkages that are suitable for chemically linked conjugates include, but are not limited to, disulfide bonds, thioether bonds, hindered disulfide bonds, and covalent bonds between free reactive groups, such as amine and thiol groups. These bonds may be produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. In some examples, several linkers can be included in order to take advantage of desired properties of each linker. Chemical linkers and peptide linkers can be inserted by covalently coupling the linker to the PTD and the POI. The heterobifunctional agents, described below, can be used to effect such covalent coupling. Peptide linkers also can be linked by expressing DNA encoding the linker and the POI; the linker and the PTD; or the PTD, linker and POI as a fusion protein. Flexible linkers and linkers that increase solubility of the conjugates are of interest in certain instances; either alone or with other linkers.

Linkers can be any moiety suitable to associate a PTD and a POI. Such moieties include, but are not limited to, peptidic linkages; amino acid and peptide linkages, such as those containing between one and 50 amino acids; and chemical linkers, such as heterobifunctional cleavable cross-linkers. Other linkers include, but are not limited to peptides and other moieties that reduce steric hindrance between the PTD and the POI, linkers that increase the flexibility of the conjugate, linkers that increase the solubility of the conjugate, or linkers that increase the serum stability of the conjugate. In some methods, where cleavage of the PTD is desired, the linkers can include intracellular enzyme substrates, photocleavable linkers and acid cleavable linkers.

The POI-PTD conjugates can be produced by genetic engineering as a fusion polypeptide that includes the PTD and the POI which can be expressed in suitable host cells. Fusion polypeptides, as described herein, can be formed and used in ways analogous to or readily adaptable from standard recombinant DNA techniques. Accordingly, provided herein are nucleic acid molecules and expression vectors comprising a nucleic acid encoding a PTD and the POI. Any convenient expression vector, as well as recombinant DNA methods and methods for making and using expression vectors, may be employed. If desired, one or more amino acids, i.e. linker peptides, can additionally be inserted between the first peptide domain comprising the PTD and the second polypeptide domain comprising the POI.

The PTD may be conjugated to the POI in a manner that does not affect the activity of the POI. The PTD can be coupled directly to the POI on one of the terminal ends (N or C terminus) or on a selected side chain of one of the amino acids of the POI. The PTD also can be coupled indirectly to the POI by a connecting arm, or spacer, to one of the terminal ends of the peptide or to a side chain of one of the amino acids.

Distributed Protein Transduction domains (PTDs)

As mentioned above, also of interest as protein transduction domains in the transduction proteins are distributed PTDs. By distributed PTD is meant a domain or region of the transduction protein that confers protein transduction capability onto the transduction protein, where protein transduction is the translocation of a protein across the cell membrane of a cell (as defined above). As the protein transduction domain is distributed, it is made up of multiple non-sequential amino acid residues that, upon folding of the transduction protein into a three-dimensional structure, make up a “basic-patch” on the surface of the protein that imparts protein transduction activity to the transduction protein. As such, the distributed PTD arises from the interaction of multiple non-sequential residues which, when the protein assumes a tertiary structure, are part of a basic patch. The multiple non-sequential residues are interspersed among the residues of the POI domain. A “basic patch” is a surface region of a folded protein that comprises 3 or more, such as 5 or more, including 10 or more % (by number) basic amino acid residues, i.e., histidine (H), lysine (K) or arginine (R). The total number of basic residues in a given basic patch may vary, ranging in some instances from 2 to 50, such as 3 to 30 and including 5 to 20. The surface area of the basic patch may vary, ranging in some instances from 5 to 10,000 Å², such as 25 to 100 Å². As the basic patch of the distributed PTD arises from non-sequential amino acid residues, at least some of the residues that participate in (i.e., are members of) the basic batch are non-sequential in the primary sequence of the POI domain. As such, two or more residues present in the basic patch of the distributed protein transduction domain may be separated from each other in the primary sequence by a region or domain that is 1 or more residues long, such as 2 or more residues long, including 3 or more residues long, e.g., 4 or more, 5 or more, 10 or more residues long. The number of pairs of residues that participate in the basic patch and are separated in the primary sequence by one or more intervening residues may vary, where the number may be 2 or more, 3 or more, 4 or more, 5 or more, 10 or more, etc. As such, the distributed PTDs are distinguished from “cannonical” protein transduction domains which are made of up sequential residues in the primary sequence of the protein in which that are found. Therefore, the distributed PTDs may be viewed as non-cannonical protein transduction domains.

As reviewed above, the POI may vary widely. Proteins of interest may include or be modified to include a distributed protein transduction domain, e.g., as described above. Proteins may be modified in a number of different ways, e.g., directed mutation, to include a distributed protein domain.

By way of example only, a POI may be a transcription modulator. Examples of transcription modulators include, but are not limited to: Tet-Off, Tet-Off Advanced, Tet-On, Tet-On Advanced or Tet On-3G transcription modulator (All of which are available from Clontech Laboratories, Mountain View, Calif.). In these instances, the transcription modulator domain may include one or more point mutations as compared to the Tet-Off, Tet-Off Advanced, Tet-On, Tet-On Advanced or Tet On-3G transcription modulators, which mutations give rise to the distributed protein transduction domain. In some instances, the point mutation is a mutation according to the formula XnY, where X is any surface-facing amino acid residue of neutral or negative charge (e.g., A, Q, E, C, etc), n is the position of the amino acid as numbered from the N-terminus and Y is H, K or R. In certain embodiments, the residue that is changed according to the above formula is chosen such that it is near in space to another positive charge, so that by changing Xn to Yn, the region of surface charge locally increases. Specific point mutations of interest include, but are not limited to: Q32R, C88R, Al 18R, E128R, C195R, Q32K, C88K, A118K, E128K, C195K. Of the above specific point mutations, a given tet transcription modulator of the invention may include 1 or more, such as 2 or more, including 3 or more, 4 or more, 5 or more, etc. of the listed point mutations.

Transduction proteins that include a distributed PTD may be fabricated using any convenient protocol. Nucleic acids encoding a parent transduction protein may be mutated using any convenient protocol to generate targeted changes in the sequence of the encoded protein to provide the desired distributed PTD. The DNA sequence or protein product of such a mutation may be substantially similar to the sequences provided herein, e.g., will differ by at least one nucleotide or amino acid, respectively, and may differ by at least two but not more than about thirty nucleotides or ten amino acids. The sequence changes may be substitutions, insertions, deletions, or a combination thereof. Deletions may further include larger changes, such as deletions of a domain or exon, e.g. of stretches of 10, 20, 50, 75, 100, 150 or more amino acid residues, where such embodiments are, in some intances, outside of the ranges provided above with respect to difference of no more than ten amino acids. Techniques for in vitro mutagenesis of cloned genes are known. Examples of protocols for site specific mutagenesis may be found in Gustin et al. (1993), Biotechniques 14:22; Barany (1985), Gene 37:111-23; Colicelli et al. (1985), Mol. Gen. Genet. 199:537-9; and Prentki et al. (1984), Gene 29:303-13. Methods for site specific mutagenesis can be found in Sambrook et al., Molecular Cloning: A Laboratory Manual, CSH Press 1989, pp. 15.3-15.108; Weiner et al. (1993), Gene 126:35-41; Sayers et al. (1992), Biotechniques 13:592-6; Jones and Winistorfer (1992), Biotechniques 12:528-30; Barton et al. (1990), Nucleic Acids Res 18:7349-55; Marotti and Tomich (1989), Gene Anal. Tech. 6:67-70; and Zhu (1989), Anal Biochem 177:120-4.

Further examples of distributed PTDs are reported in U.S. application Ser. No. 13/303,652; the disclosure of which is herein incorporated by reference.

### Additional Domains

In some instances, the transduction protein may include one or more additional domains in addition to the POI and PTD domains. For example, the transduction protein may include a protein tag domain, e.g., a tag sequence which serves as a purification tag for the POI. Any convenient tag sequences may be employed, including but not limited to those described in U.S. Pat. No. 7,176,298 and United States Patent Application Publication No. 20090023898; the disclosures of which are herein incorporated by reference. Specific tag sequences of interest include, but are not limited to: 6×His tags, 6×HN tags, etc. When present, such tags may vary in length, and in some instances range in length from 5 to 500 amino acids, such as 5 to 100 amino acids, including 6 to 12 amino acids. The tag may be positioned at any convenient location in the POI. Another optional domain of interest is a spacer domain. Spacer domains, when present, may vary in length, ranging in some instances from 2 to 50, such as 5 to 15 amino acids. While the sequence of a spacer domain may be any convenient sequence, in some instances the sequence is a poly-Alanine sequence, a poly glycine sequence, or a mixed amino acid sequence. As with tag domains, spacer domains may be positioned at any convenient location in the transduction protein.

### Transduction Methods

As summarized above, aspects of the invention include transducing a target cell with a transduction protein, e.g., as described above. Accordingly, aspects of the invention further include methods of transducing a target cell, where the target cell may be in vitro or in vivo. The target cell that is transduced with the transduction protein may vary widely. Target cells may be single cells, cell lines or components of a multi-cellular organism. In some instances, the target cell is a eukaryotic cell. Some examples of specific cell types of interest include, but are not limited to: bacteria, yeast (e.g., S. cerevisiae, S. pombe, P. pastoris, K. lactis, H. polymorpha); fungal, plant and animal cells. Target cells of interest include animal cells, where specific types of animal cells include, but are not limited to: insect, worm or mammalian cells. Various mammalian cells may be used, including, by way of example, equine, bovine, ovine, canine, feline, murine, non- human primate and human cells. Among the various species, various types of cells may be used, such as hematopoietic, neural, glial, mesenchymal, cutaneous, mucosal, stromal, muscle (including smooth muscle cells), spleen, reticulo- endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells).

As desired, target cells may be transduced in vitro or in vivo. For target cells that are transduced in vitro, such cells may ultimately be introduced into a host organism. Depending upon the nature of the cells, the cells may be introduced into a host organism, e.g. a mammal, in a wide variety of ways. Hematopoietic cells may be administered by injection into the vascular system, there being 10⁴ or more cells and in some instances10^{l0} or fewer cells, such as I0⁸ or fewer cells. The number of cells which are employed will depend upon a number of circumstances, the purpose for the introduction, the lifetime of the cells, the protocol to be used, for example, the number of administrations, the ability of the cells to multiply, the stability of the therapeutic agent, the physiologic need for the therapeutic agent, and the like. Alternatively, with skin cells which may be used as a graft, the number of cells would depend upon the size of the layer to be applied to the burn or other lesion. For myoblasts or fibroblasts, the number of cells may be 10⁴ or greater and in some instances 10⁸ or less, where the cells may be applied as a dispersion, generally being injected at or near the site of interest. The cells may be in a physiologically-acceptable medium.

Transduction methods of the invention include contacting the target cell with a suitable amount of the transduction protein and nucleic acid transfection agent under conditions sufficient for the transduction protein to transduce the target cell, i.e., for the transduction protein to be internalized by the target cell. In these embodiments, the target cell is contacted with the transduction protein under transduction conditions. In certain instances, an amount of transduction protein is contacted with an amount of target cells under cell culture conditions and incubated for a sufficient period of time for transduction to occur. In some instances, an amount of transduction protein ranging from 1 molecule per cell to 1×10E¹² molecules per cell, such as 7×10E⁷ molecules per cell to 7×10E⁸ molecules per cell is contacted with an amount of target cells ranging from 1 to 50 million, such as 10,000 to 100,000 cells. Any convenient culture medium may be employed, where culture media of interest include, but are not limited to: RPMI, DMEM, OptiMEM and the like. The cells and transduction protein may be incubated for varying amounts of time, e.g., from 5 minutes to 5 days, such as 1 to 4 hours, at various temperatures, e.g., ranging from 4 to 42, such as 30 to 37° C.

As summarized above, during transduction the target cell is contacted with the transduction protein in the presence of a nucleic acid transfection reagent. Nucleic acid transfection reagents are chemicals that enhance transduction, e.g., by 2 fold or more, e.g., 3 fold or more, including 5 or 10 fold or more. Transfection reagents suitable for use in methods of the invention may vary. Transfection reagents of interest include, but are not limited to chemical based transfection reagents, including but not limited to: cyclodextrin based reagents, polymer based reagents (e.g., DEAE-dextran or polyethylenimine based reagents), dendrimer based reagents, liposomes based reagents (e.g., cationic polymer based reagents), etc. Nucleic acid transfection reagents of interest include, but are not limited to: Xfect™ transfection reagent from Clontech Laboratories, Lipofectamine LTX transfection reagent from Life Technologies, Lipofectamine 2000 transfection reagent from Life Technologies, SiQuest transfection reagent from Mirus, Transit-siQuest transfection reagent, Transit-TKO transfection reagent, Transit-LTI transfection reagent, Transit-Jurkat transfection reagent, Transit-2020 transfection reagent; chloroquine, PEG, etc.

For in vitro applications, the nucleic acid transfection reagent may be included in the culture medium at any convenient concentration, where in some instances concentrations range from 0.001 mg/mL to 0.1 mg/mL, such as 0.0025 to 0.036 mg/mL and including 0.001 to 0.01 mg/mL. The transfection agent may be included in the culture medium before, at the same time as or after the transduction protein is provided in the medium, such that any convenient sequence of contact of the transduction protein and transfection reagent with the target cell may be employed.

### Utility

Methods and compositions of the invention find use in any application where it is desirable for a cell to internalize a protein. In other words, the methods and compositions described herein find use any protein transduction application. Methods and compositions of the invention find use in both in vitro and in vivo applications. Applications in which the methods and compositions find use include, but are not limited to research, diagnostic and therapeutic applications. Specific types of applications of interest include, but are not limited to: the study of cellular development and differentiation in eukaryotic cells, plants and animals; in vitro protein production; in vivo protein production; imaging of regulated gene expression in vivo; animal models of human disease; production of stable cell lines; expression of inhibitor RNA; drug screening, gene therapy; etc. Applications in which the methods and compositions of the invention find use are further described in U.S. Patent Nos. U.S. Pat. Nos. 5,888,981; 5,866,755; 5,789,156; 5,654,168; 5,650,298; 6,004,941; 6,271,348; 6,271,341; 6,783,756; 5,464,758; 6,252,136; 5,922,927; 5,912,411; and 5,859,310; as well as United States Published Patent Application No. 20090257985; the disclosures of the specific applications disclosed in these publications being specifically incorporated herein by reference.

### Kits

Additional aspects of the invention include kits, e.g., for use in transduction applications. Kits of the invention at least include a nucleic acid transfection agent, e.g., as described above. In some instances, the kits may further include a transduction protein, where the protein may include a distinct PTD or distributed PTD. Alternatively, kits may include a vector configured for expressing a transduction protein of interest in a host cell. Such a vector may include an expression cassette which has a distinct PTD encoding domain and a cloning site (e.g., in the form of a restriction site, such as a multiple cloning site (MCS)), for receiving a POI coding sequence. The vector may further include one or more additional components, e.g., a promoter, selectable marker, operably linked to the other components of the vector, etc. In addition to the nucleic acid transfection agent and optional components described above, the kits may include yet more additional components. Additional components that may be present in the kits include, but are not limited to: a host cell line; a control cell line; etc. The various reagent components of the kits may be present in separate containers, or some or all of them may be pre-combined into a reagent mixture in a single container, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), etc., on which the information has been recorded. Yet another form of these instructions that may be present is a website address which may be used via the internet to access the information at a removed site.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); nt, nucleotide(s) and the like.

### Experimental

### EXAMPLE

### Use of DNA Transfection Reagents Enhances Transduction

During the development of the Tet Express protein, we noticed that co-addition of several different DNA transfection reagents greatly enhanced the luciferase activity in a Tet reporter cell line. Tet Express protein has the sequence:

(SEQ ID NO: 01)

MSRLDKSKVINSALELLNEVGIEGLTTRKLARKLGVEQPTLYWHVKNKRA
LLDALAIEMLDRHHTHFCPLEGESWQDFLRNNAKSFRRALLSHRDGAKVH
LGTRPTEKQYETLENQLRFLCQQGFSLRNALYALSAVGHFTLGCVLEDQE
HQVAKEERETPTTDSMPPLLRQAIELFDHQGAEPAFLFGLELIIRGLEKQ
LKCESGGPKLHNHNHNHNHNHNEFAAAAAAAAAGTPADALDDFDLDMLPA
DALDDFDLDMLPADALDDFDLDMLPG

Underlining denotes the transcription modulator domain (tetR), italics denotes the expression modulatory domain (VP16), the string of alanines (A) denotes the linker, and bold denotes the arginine substitutions made to improve transduction activity:

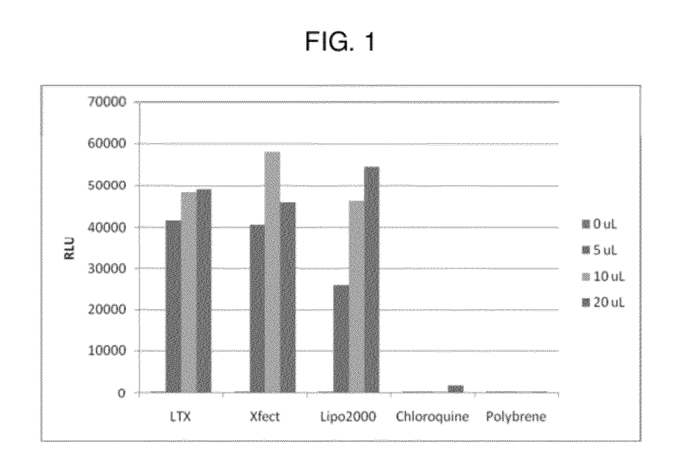

During the course of Tet Express development, we switched from transient transfection of a reporter plasmid to using a stable reporter cell line. These cells are a HeLa based cell line with a stably integrated reporter that expresses ZsGreenl and firefly luciferase under the control of a TRE-tight promoter (Clontech Laboratories, Mountain View, Calif.). This meant that we no longer needed to do DNA transfection to assay Tet Express activity. We noticed that this switch reduced the observed Tet Express activity and determined that activity could be restored by performing the transduction in the presence of certain DNA transfection reagents. The reagents that enhanced transduction best include Clontech's Xfect, Life Technologies Lipofectamine LTX and 2000 as well as Mirus' SiQuest reagent (FIGS. 1 & 2).

FIG. 1 provides the results of a luciferase assay showing enhancement of Tet Express activity. HeLa reporter cells (Clone 19) with a stable bidirectional TREtight ZsGreen/FLuc integration cassette were plated in 96 well format (15,000 cells per well). After cell attachment, 0, 5, 10 or 20 μL of transduction mix were added per well. Transduction mixes consisted of Tet Express protein in Optimem media with one of the following additions: 2% LTX, 0.6% Xfect, 2% Lipofectamine 2000 (Lipo2000), 0.2 mM chloroquine or 6 μg/mL polybrene. Luciferase assays were performed the following day.

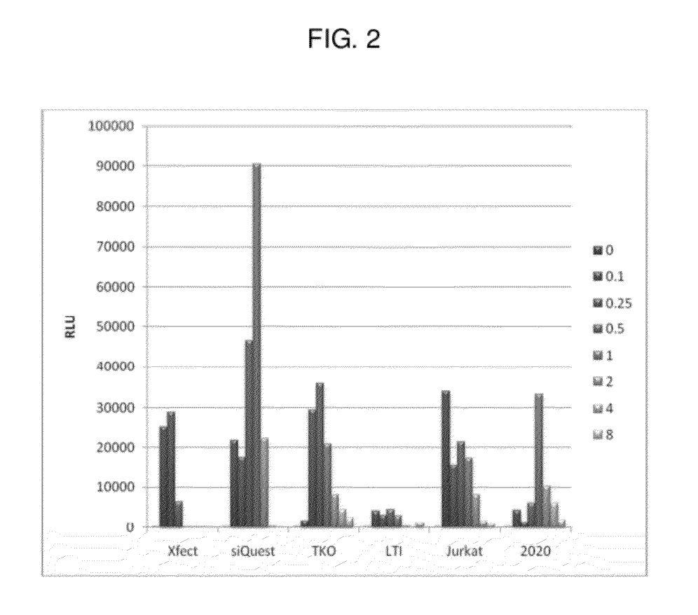

FIG. 2 provides the results of a luciferase assay showing enhancement of Tet Express activity. Clone 19 HeLa reporter cells were plated in 96 well format (15,000 cells per well). Tet Express protein (in Optimem media) was mixed with indicated amount of reagent and added to the cells. Reagents included Mirus' Transit-siQuest, Transit-TKO, Transit-LTI, Transit-Jurkat, Transit-2020 or Clontech's Xfect. Luciferase assays were performed the following day.

Other reagents that did show the enhancement effect but to a lesser degree include Mirus' TKO, LTI, Jurkat, & 2020 reagents, chloroquine, PEG. Other reagents which performed poorly, if at all, included polybrene & DMSO.

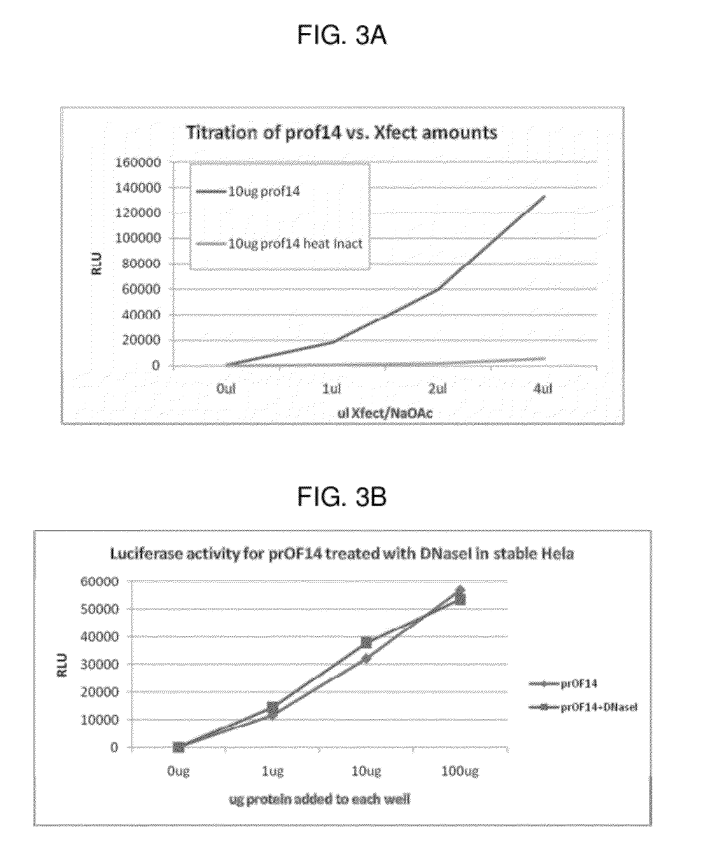

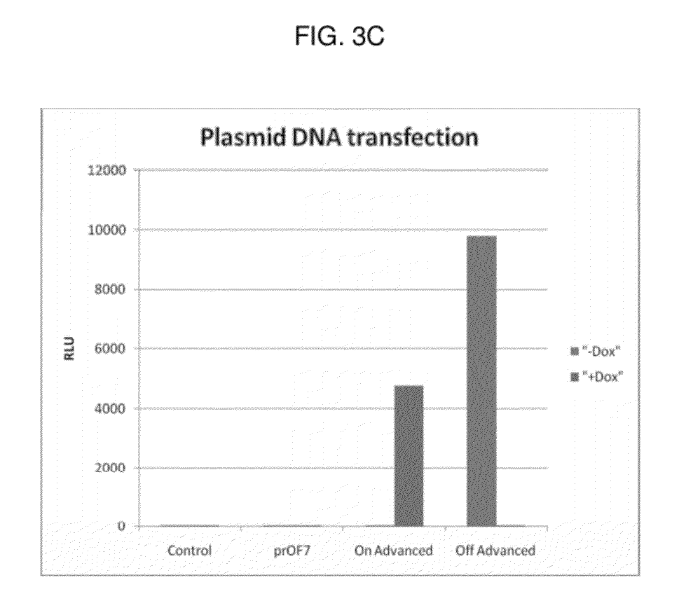

FIG. 3A provides the results of a luciferase assay using heat inactivated Tet Express. Clone 19 HeLa reporter cells were plated in 96 well format (15,000 cells per well). Ten micrograms of Tet Express or heat inactivated Tet Express (75 ° C. for 5 minutes) was mixed with either: 0, 1, 2 or 4 μL of Xfect and added to the cells. Luciferase assays were performed the following day. As illustrated in FIG. 3B, DNase treatment does not alter Tet Express activity. Clone 19 HeLa reporter cells were plated in 96 well format (15,000 cells per well). Tet Express without or with DNase I pretreatment (10 units per 150 μL Tet Express, 37° C. for 30 minutes) was mixed with Xfect and added to cells. Luciferase assay was performed the following day. In FIG. 3C., clone 19 HeLa reporter cells were plated in 12well format (50,000 cells per well). Cells were transfected with 0.5 μg of pUC19 (control), prOF7, pTet On Advanced or pTet Off Advanced plasmids with 2 μL of Lipofectamine LTX. The following day doxycyline was added to 1000 ng/mL. Luciferase assays were performed the following day.

As shown in FIGS. 3A-3C, we addressed the concern that the observed increase in luciferase activity could be due to a contaminating plasmid DNA (i.e., the bacterial expression vector for Tet Express) rather than by enhanced protein transduction. The experiments performed included:
1. Treating the Tet Express protein prep with DNase (had no effect);
2. Heat inactivating the protein prep 5 minutes at 70-75° C. (led to loss of activity);
3. Direct transfection of the bacterial expression vector encoding Tet Express into the mammalian reporter cells (no effect). The results of these experiments ruled out plasmid DNA contamination as the cause of the increased luciferase activity.

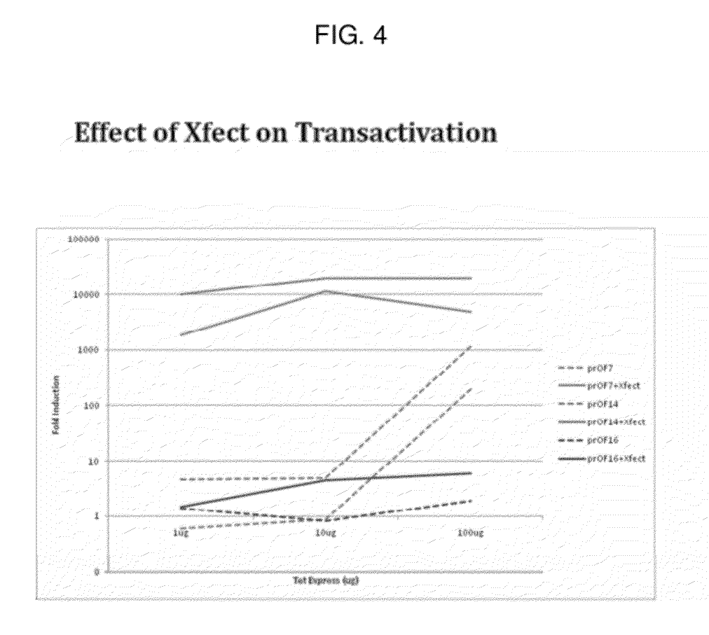

FIG. 4 illustrates the effect of Xfect (Clontech Laboratories, Mountain View Calif.) on Transactivation. Clone 19 HeLa reporter cells were plated at 50,000 cells per well in a 12 well format. Cells were treated with 0, 1, 10 or 100 μg of Tet Express (or derivative) in the presence or absence of Xfect reagent. Luciferase assays were performed the following day.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of transducing a cell with a protein, the method comprising contacting the cell with:
(a) a transduction protein comprising:
(i) a protein-of-interest (POI) domain; and
(ii) a protein transduction domain (PTD); and
(b) a nucleic acid transfection agent;
under conditions sufficient to transduce the cell with the protein.

2. The method according to claim 1, wherein the transduction protein comprises a distinct PTD.

3. The method according to claim 2, wherein the distinct PTD comprises from 5 to 100 amino acids.

4. The method according to claim 3, wherein the distinct PTD is selected from the group consisting of PTD found in TAT, VP22,antp and poly-arginine.

5. The method according to claim 1, wherein the protein comprises a distributed PTD.

6. The method according to claim 5, wherein the distributed PTD comprises a basic patch made up of 3 or more non-sequential basic amino acid residues.

7. The method according to claim 6, wherein the number percentage of basic amino acid residues in the basic patch is 30 or more.

8. The method according to claim 1, wherein the nucleic acid transfection reagent is selected from the group consisting of Xfect™ transfection reagent, Lipofectamine™ LTX transfection reagent, Lipofectamine 2000™ transfection reagent, SiQuest™ transfection reagent, Transit-siQuest™ transfection reagent, Transit-TKO™ transfection reagent, Transit-LTI™ transfection reagent, Transit-Jurkat™ transfection reagent, Transit-2020™ transfection reagent; chloroquine, PEG, and combinations thereof.

9. The method according to claim 1, wherein the method further comprises evaluating the cell to determine whether the cell has been transduced with the transduction protein.

10. The method according to claim 1, wherein the method is an in vitro method.

11. A transduction system comprising:
(a) a host cell;
(b) a transduction protein comprising:
(i) a POI domain; and
(ii) a PTD; and
(c) a nucleic acid transfection agent.

12. The system according to claim 11, wherein the transduction protein comprises a distinct PTD.

13. The system according to claim 12, wherein the distinct PTD comprises from 5 to 100 amino acids.

14. The system according to claim 13, wherein the distinct PTD is selected from the group consisting of PTD found in TAT, VP22, antp and poly-arginine.

15. The system according to claim 11, wherein the transduction protein comprises a distributed PTD.

16. The system according to claim 15, wherein the distributed PTD comprises a basic patch made up of 3 or more non-sequential basic amino acid residues.

17. The system according to claim 16, wherein the number percentage of basic amino acid residues in the basic patch is 30 or more.

18. The system according to claim 11, wherein the nucleic acid transfection reagent is selected from the group consisting of from the group consisting of Xfect™ transfection reagent, Lipofectamine™ LTX transfection reagent, Lipofectamine 2000™ transfection reagent, SiQuest™ transfection reagent, Transit-siQuest™ transfection reagent, Transit-TKO™ transfection reagent, Transit-LTI™ transfection reagent, Transit-Jurkat™ transfection reagent, Transit-2020™ transfection reagent; chloroquine, PEG, and combinations thereof.

19. A kit comprising:
(a) at least one of:
(i) a vector comprising an expression cassette comprising a distinct PTD encoding element; and a cloning site; and
(ii) a transduction protein; and
(b) a nucleic acid transfection agent.

20. The kit according to claim 19, wherein the nucleic acid transfection reagent is selected from the group consisting of from the group consisting of Xfect™ transfection reagent, Lipofectamine™ LTX transfection reagent, Lipofectamine 2000™ transfection reagent, SiQuest™ transfection reagent, Transit-siQuest™ transfection reagent, Transit-TKO™ transfection reagent, Transit-LTI™ transfection reagent, Transit-Jurkat™ transfection reagent, Transit-2020™ transfection reagent; chloroquine, PEG, and combinations thereof.
